(19) United States
(12) Patent Application Publication
(10) Pub. No.: US 2021/0405007 A1
(43) Pub. Date: Dec. 30, 2021

(54) LOW-FLOW SIZE-SELECTIVE INLET FOR AIR QUALITY SENSORS AND AIR QUALITY SENSOR

(71) Applicant: US Environmental Protection Agency, Office of General Counsel - General Law Office, Washington, DC (US)

(72) Inventors: Paul Alan Solomon, Henderson, NV (US); Lara A. Gundel, Berkeley, CA (US)

(73) Assignee: the United States Government, as represented by the Administrator of the U.S. Environmental Protecti, Washington, DC (US)

(21) Appl. No.: 16/917,889

(22) Filed: Jun. 30, 2020

(51) Int. Cl. G01N 33/00; G01N 1/24; G01N 1/28; G01G 3/13

(52) U.S. Cl. CPC G01N 33/0031; G01N 1/24; G01N 2001/245; G01G 3/13; G01N 1/28

(57) ABSTRACT

An inlet or primary particle size fractionator for a direct-reading PM_{2.5} mass sensor described herein may remove atmospheric particles of a given size, such as particles greater than the inlet cut point (e.g., having a 10 μm AD cut point) and may transport particles less than the cut point to a mass sensing element or a secondary particle size fractionator (e.g., having a 2.5 μm AD cut point). The inlet may have a flow rate range of between 1 mL/min and 50 mL/min (or higher flow rates depending on the application). The inlet may include a virtual impactor (VI), real impactor, cyclone, or virtual cyclone (VC). A sensing element may measure particle mass below the primary particle size fractionator (e.g., 2.5 μm AD particles with a 10 μm AD cut point inlet) and/or between the size range of the primary and secondary particle size fractionators (e.g., between 2.5 μm and 10 μm AD, or coarse particles).

### BACKGROUND

The availability of small, portable and inexpensive air pollution particle and gas sensors, as well as recent developments in electrical engineering and computer science, provide opportunities for simultaneous data-rich continuous monitoring of air pollutants over wide areas of space and time.

Various particle sizes found in ambient outdoor or indoor air samples may be of interest.

### BRIEF DESCRIPTION OF THE SEVERAL VIEWS OF THE DRAWINGS

The novel features of the disclosure are set forth in the appended claims. However, for purpose of explanation, several embodiments are illustrated in the following drawings.

FIG. 1 illustrates a schematic block diagram of an exemplary low-flow air quality sensing system of some embodiments;

FIG. 2 illustrates a front elevation view of a virtual impactor (VI) used by some embodiments as an extra low maintenance inlet for an air quality sensor;

FIG. 3A illustrates a front elevation view of a cyclone and collection well used as an air quality sensor inlet by some embodiments;

FIG. 3B illustrates a front elevation view of a virtual cyclone and output fan or blower used as an extra low maintenance air quality sensor inlet by some embodiments;

FIG. 4 illustrates a front elevation view of a real impactor of some embodiments used as an air quality sensor inlet by some embodiments;

FIG. 5A illustrates a front elevation view of a secondary fractionator of some embodiments;

FIG. 5B illustrates a side elevation view of an alternative secondary fractionator of some embodiments;

FIG. 6 illustrates a front elevation view of a primary fractionator including a collection and/or sensing element and a virtual cyclone of some embodiments;

FIG. 7 illustrates a flow chart of an exemplary process that that generates mass measurements for multiple particle sizes and/or size ranges; and

FIG. 8 illustrates a schematic block diagram of one or more exemplary devices used to implement various embodiments.

### DETAILED DESCRIPTION

The following detailed description describes currently contemplated modes of carrying out exemplary embodiments. The description is not to be taken in a limiting sense, but is made merely for the purpose of illustrating the general principles of some embodiments, as the scope of the disclosure is best defined by the appended claims.

Various features are described below that can each be used independently of one another or in combination with other features. Broadly, some embodiments generally provide air quality sensors that directly or indirectly measure different size fractions of particulate matter (PM).

A highly flexible, low-cost, low power, portable, wireless, real-time meso- and micro-scale direct-reading PM mass sensor that measures the mass of particles (directly or indirectly) at very low flow rates (e.g., <50 mL/min, however higher flowrates may apply depending on the application) that allows for a better understanding of the spatial and temporal scale of air pollutants that adversely impact public health, global warming, and regional climate change. Sensors may be able to measure multiple defined size fractions of PM simultaneously and at high time resolution. Further, the sensors may be able to measure PM across a range or group of sizes either directly or by calculating a difference between two measured size fractions.

PM size fractions may include, for example, PM₁₀ (particles less than 10 μm in aerodynamic diameter (AD)), PM₁₅ (particles less than 15 μm AD), PM₄ (particles less than 4 μm AD), PM_{2.5} (particles less than 2.5 μm AD), PM_{1.0} (particles less than 1 μm AD), or PM_{0.1} (particles less than 0.1 μm AD). PM size fractions may include particles in size ranges between, for example, 2.5 μm and 10 μm AD. Such a range may be referred to as “coarse” PM (or “PM₅”). Particle size ranges may be referred to as “PM_{X-Y}”, where (Y represents the particle size greater than the cut point of the secondary fractionator and X represents particles less than the cut point of the primary fractionator). A “cut point” is defined as the 50% collection efficiency of the particle size of interest based on a calibration of the device with particles of known size. Different embodiments may be associated with various different particle sizes (i.e., less than the cut point size, e.g., 10 μm, 2.5 μm) or size ranges, such as particles between the upper (primary size fractionator cut point size) and lower (secondary size fractionator cut point size), including those listed or described herein and/or additional or other sizes or size ranges, as appropriate.

Due to small size, low cost, and low power consumption, the sensor (including fractionators and sensing elements) of some embodiments may be widely distributed to obtain PM mass measurements in multiple size ranges at a much finer spatial resolution than current solutions. One or multiple sensing elements associated with one or more size fractionating sensors may communicate PM mass measurement data and may be linked to various networks such that data is collected and made available via the Cloud. Such data collection and provision may allow real-time evaluation of personal, local, regional, and global pollutant variability in space and time.

The sensors may be utilized in indoor and outdoor environments. Sensors may be included in stationary or mobile platforms (e.g., personal devices, ground-based vehicles, aerial devices or platforms, etc.) to collect data regarding personal exposure at locations or areas of interest (e.g., on and near roads, within residential communities, near industrial areas, etc.). Sensors may be deployed in aerial platforms in order to better understand the vertical distribution of PM size fractions, allowing analysis of transport patterns at elevation. Sensors of some embodiments may be linked to external systems or devices, such as wearable sensors that monitor individual health outcomes or attributes associated with air pollution. Sensors may be manufactured using one or more appropriate processes (e.g., micro- or macro-machining, microfabrication, 3-D printing, etc.).

Some embodiments may measure particle sizes such as, for example PM_{1.0} or PM_{0.1}, with a focus on the smallest particles, which are believed to be of most harm to human health. Various different sets of sizes (defined by the fractionator cut point) or sets of ranges of sizes (defined by the difference between size ranges of different cut points) of particles may be measured at the same sensor sites, using one or more sensors. Such broad measurement capability is especially important at near- or on-roadway sites and mobile platforms related to mobile sources and near other sources to better understand their impacts. Data collected from such sensors may provide improved estimates of personal exposure allowing for improved analysis of links between PM sources and adverse health effects.

Some embodiments provide both a direct measure of regulated PM size ranges (e.g., PM₁₀ and PM_{2.5}), as well as a direct and/or calculated measure of PM_{c}. These inlets may be applicable to some sensors that measure PM indirectly using light scattering to minimize contamination of the optical sensing elements or gas sensors to minimize contamination of the sensing element (e.g., an electrochemical cell). Other size ranges (e.g., PM_{4.0}, PM_{1.0}, or size ranges between PM₁₀ and PM_{4.0}, or between PM_{1.0} and PM_{2.5}) may also be measured using the particle size fractionators described herein by setting the inlet cut point (i.e., a particle size associated with a fifty percent collection efficiency) at a desired value (e.g., 1 μm AD, 4 μm AD, 10 μm AD, etc.) and the fractionator cut point at a desired value (e.g., 2.5 μm AD, 1 μm AD, or 0.1 μm AD). While PM₁₀ may be the primary inlet target cut point, some embodiments may have smaller inlet cut points, while other embodiments may have larger inlet cut points. For example, some embodiments may have an inlet cut point of 15 μm AD. Some embodiments, using a virtual cyclone, may provide a cut point as high a 100 μm AD.

Some embodiments may be optimized for flowrates from 1 mL/min to 50 mL/min; higher flowrates may apply depending on the application. For the smallest possible sensor, a real impactor or cyclone for PM₁₀ may operate at 1 mL/min, whereas a virtual impactor or virtual cyclone may operate at a slightly higher flow rate (e.g., four to ten percent higher for each fractionator) to account for the portion of flow that follows the minor channel of, for example, the primary and secondary fractionator when each use a virtual cyclone and/or virtual impactor.

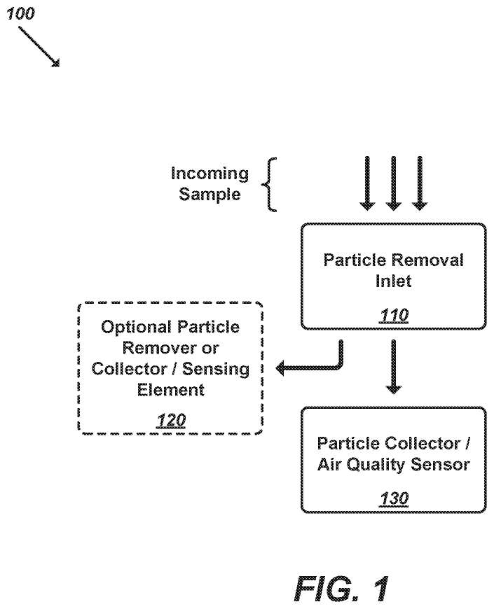

FIG. 1 illustrates a schematic block diagram of an exemplary air quality sensing system 100 that may include a particle removal inlet 110, an optional particle remover, particle collector, and/or sensing element 120, and a particle collector, air quality sensor, and/or secondary fractionator 130 of some embodiments. In some embodiments, some or all of the components of air quality sensing system 100 may be included in an integrated sensing device. In some embodiments, the elements of system 100 may be included in a single housing or packaging (and/or multiple distinct elements coupled together), which may be referred to as an air quality “sensor” 100. Air quality sensor 130 may include multiple separate devices, components, etc. or may be implemented as a stand-alone device that houses or otherwise includes the various elements. As shown, the particle removal inlet or “primary particle size fractionator” 110 may include an output connected to an optional particle collector and/or sensing element 120 that collects and/or directly measures a first fraction of PM, and an output connected to the air quality sensor 130. If the optional particle collector and/or sensing element 120 is not included, particles may be removed through a fan or pump.

The air quality sensor 130 may output a voltage or PM mass data in near-real or real time via various electronics and communication protocols, including wireless communications to the cloud. The air quality sensing system 100 may include various other components as described below, including embedded sensors for temperature, relative humidity, pressure, flow rate, feedback to control flow rate (e.g., by adjusting fan or pump speed), and others that can provide data to allow the determination of mass concentration from the output voltage. The air quality sensing system 100 (or elements thereof, such as air quality sensor 130) may include any or all elements described below in reference to device 800.

The particle removal inlet 110 may perform fractioning of particles into two distinct size ranges (e.g., larger than PM₁₀ and smaller than PM₁₀). Throughout this disclosure, inlets may be described by reference to particle size(s) removed and/or collected (e.g., a “PM₁₀ inlet” may separate and remove particles larger than 10 μm AD). Particles larger than the specified cut point may be rejected (i.e., collected, diverted, or otherwise removed) within or at the minor flow exit of the inlet and the resulting stream into the optional particle collector and/or sensing element 120 may include particles larger than the specified cut point, while the stream into the air quality sensor 130 may include only particles smaller than the specified cut point. Particle remove or filtering may have an associated collection efficiency (e.g., fifty percent). The cut point may include various tolerances, such as, ±1 μm, for a 10 μm cut point, plus/minus ten percent of the cut point AD, etc., among other appropriate tolerances that may be applicable (e.g., collection efficiency tolerances).

The inlet 110 may include a fractionator having a plate (i.e., a real impactor), a collection cone (i.e., a round-nozzle or rectangular jet virtual impactor (VI)), a cyclone, a virtual cyclone, a filter, and/or other appropriate particle sorting or particle removing elements. The inlet 110 may also include a circular rain guard with a bug screen to minimize rain, snow, bugs from entering the inlet and to reduce the influence of wind on the collection of PM into the inlet. Some embodiments may include a slit virtual impactor as the particle removal inlet 110, where the length of the slit is much longer than the width of the slit. A circular slit around the circumference of the inlet may best reduce influence from wind direction in conjunction with a circular wind guard.

Major channels (i.e., channels including particles smaller than the inlet cut point) may carry from eighty-five percent to ninety-six percent of the input flow (e.g., ninety percent) coming into the inlet 110 for inlets utilizing virtual impactors or virtual cyclones. The minor channel (i.e., a channel including particles larger than the inlet cut point) may receive the remainder of the flow (i.e., four percent to fifteen percent), such as, for example, ten percent of the flow. Such a split (ninety percent to ten percent for major to minor flow) concentrates the larger size particles by a factor of ten. Other splits may be useful for concentrating the coarse particles to a higher degree. For example, a four percent minor channel split rate may concentrate the larger particles by a factor of twenty-five. Concentration occurs because the portion of input flow that passes through the minor channel (e.g., ten percent or four percent) includes all of the larger particles from the input flow.

For embodiments that use a real impactor, the particles may be impacted onto a physical surface (e.g., a plate). The fine particles (i.e., particles sized smaller than the cut point size) may follow the major flow (at some specified collection efficiency, such as fifty percent) around the plate and the larger particles (i.e., particles sized larger than the cut point size) may be impacted onto the plate. The plate may include one or more sensing elements (e.g., a quartz crystal (QC), solid mounted resonator (SMR), or other resonator whose frequency changes as particle mass increases on the resonator). In some embodiments, the cut point is 10 μm AD and the slope of the collection efficiency curve defines the efficiency of the cut point. Particles less than 10 μm AD (at fifty percent collection efficiency in some embodiments) may then flow into an air quality sensor 130 (and/or other component, such as a particle size fractionator) where the particles may be collected directly onto a piezoelectric resonator (e.g., a QC or SMR), or other resonator whose frequency changes as particle mass increases on the resonator) or other direct-mass or indirect-mass sensor. If collected using a real impactor, a direct measure of PM₁₀ may be provided, where no concentration occurs with real impactors.

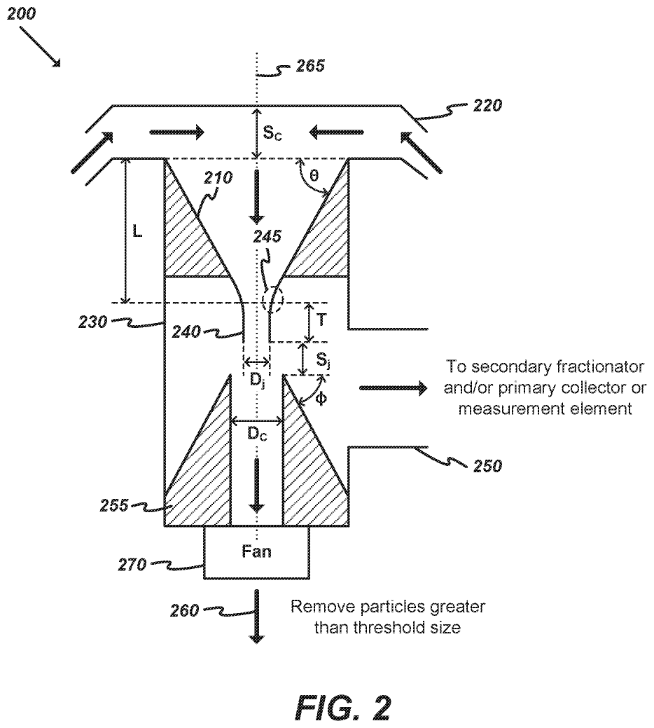

Some embodiments may provide a PM₁₀ inlet 110 with a sharp collection efficiency curve having a slope of 1.5 or better (calculated based on a slope of the collection efficiency curve, where the x-axis indicates particle diameter, and the y-axis indicates percent collection efficiency). Several embodiments of a PM₁₀ inlet that remove particles larger than 10 μm AD with a fifty percent collection efficiency are described below in reference to FIG. 2-FIG. 4.

Several embodiments are also described for the collection of particles in the size range between 2.5 μm AD and 10 μm AD, although other size ranges may be collected depending on the cut point for the inlet 110 and a secondary fractionator 130 (or additional fractionators). Such collection may include both real impaction of PM_{c} onto a quartz crystal microbalance (QCM) either associated with a direct-reading PM_{2.5} mass sensor or utilization of a virtual cyclone operated at flow rates from 1 mL/min to 50 mL/min (or higher, depending on the application). Because such sampling relies on inertia or centrifugal force, sensors of some embodiments may be positioned in a vertical orientation with gravity applying force from the inlet toward the collection substrate, especially for particles greater than 3 μm AD. Such vertical orientation may provide the sharpest slope and lowest wall losses. Real impactors may also be positioned in various other orientations (e.g., horizontal) where particles less than the cut point would exit out of the primary output of the real impactor and particles larger than the cut point may impact on a flat plate or other appropriate element and fall by gravity to the bottom of the impactor, providing a low maintenance impactor.

Returning to FIG. 1, inlet 110 may be followed by an optional particle collector and/or sensing element 120. The optional particle collector and/or sensing element 120 may serve as a particle repository and/or may be capable of measuring various attributes of the collected particles (e.g., using a resonator or other sensing element, such as a light scattering sensing element, to determine particle mass, density, chemical composition, etc.). In some embodiments, the optional particle collector and/or sensing element 120 may include an exhaust or other appropriate output that may disperse or otherwise dispose of particles above the cut point, rather than collecting, measuring, or otherwise processing such particles (e.g., those particles above 10 μm AD for a PM₁₀ inlet). The inlet 110 may be microfabricated or meso-fabricated, including 3-D printed.

In some embodiments, air quality sensing system 100 may include one or more secondary particle size fractionators that each may have a smaller cut point than the inlet 110 and may include a real impactor, VI, cyclone, virtual cyclone, a filter, and/or other appropriate particle sorting or particle removing elements. Such secondary particle size fractionators may have a cut point defined for the collection and/or measurement of, for example, PM₄, PM_{2.5}, or PM_{1.0}, and/or other size fractions. The secondary fractionator may be microfabricated or meso-fabricated, including 3-D printed. Thus, using the inlet 110 (or “primary” fractionator) and secondary fractionators, some embodiments may provide direct measurement in real-time of coarse particles (i.e., particles sized greater than the cut point of the secondary fractionator and less than the cut point of the inlet 110) in various size ranges (e.g., between 2.5 μm and 10 μm AD, between 4 μm AD and 10 μm AD, between 1.0 μm AD and 2.5 μm AD, and/or other appropriate ranges).

In some embodiments, particles less than the inlet or “primary” cut point (e.g., 10 μm AD) may enter the secondary particle size fractionator. The secondary particle fractionator then may further divide particles less than the inlet cut point of the primary fractionator into two groups of particles—a first group of particles sized in the range between the secondary cut point (e.g., 2.5 μm AD) and the primary cut point of the inlet (e.g., 10 μm AD) and a second group of particles sized less than a secondary cut point (e.g., 2.5 μm AD, which is also let than the primary cut point). Particles sized smaller than the secondary cut point (e.g., PM_{2.5}) may be collected by deposition onto sensing elements. Such sensing elements may include resonators such as film bulk acoustic resonators (FBARs) and/or other appropriate sensing elements. Particles sized between the primary and secondary cut points may be collected on sensing elements (e.g., resonators, such as a QCM or SMR, as lower frequency resonators may be more suited to measurements of larger particles than ultra-sensitive resonators such as FBARs). In addition, FBARs quickly fail if deposited particles have diameters the same order of magnitude as the thickness of the piezoelectric layer of aluminum nitride on the FBAR. Such resonators or other sensors may be included at the secondary particle size fractionator and/or secondary sensing element. Generally, FBARs may be most suitable for particles≤2.5 μm AD and the QC and SMR may be most suitable for particles>2.5 μm AD.

In some embodiments, air quality sensing system 100 may include one or more secondary collectors and/or sensing elements that may serve as particle repositories and/or may be capable of measuring various attributes of the collected particles (e.g., using a resonator or other sensor to determine particle mass, density, chemical composition, etc.). Some embodiments may include two secondary collectors and/or sensing elements, both associated with the secondary particle size fractionator, where particles having been separated into two particle size ranges may be collected and measured by two separate sensing elements. Particles sized above the secondary cut point may be collected in one secondary collector or sensing element, while particles sized below the secondary cut point may be collected in the other secondary collector or sensing element (where particles in both secondary collectors may be sized below the primary cut point of the inlet 110). Any number of additional particle size fractionators and/or collectors/sensing elements may be included.

Elements of air quality sensing system 100, such as air quality sensor 130, may be able to communicate among the other elements of air quality sensing system 100 and may at least partly direct the operations of various components associated with the air quality sensing system 100 or components thereof (e.g., fans or pumps, valves, user interface elements, communication modules, etc.). Examples of such components will be described in more detail in reference to device 800 below. In some embodiments, elements of air quality sensing system 100 may be able to perform various calculations based on data received from the optional particle collector and/or sensing element 120 and/or air quality sensor 130. For instance, the air quality sensing system 100 may determine a mass or density of collected particles. As another example, the air quality sensing system 100 may calculate a difference between calculated particle masses to determine an amount (e.g., a mass) of particles within a specified range. As still another example, the measurement module may normalize data based on particle concentration of a sample portion (e.g., for a minor channel having a particle concentration ten times that of a collected sample, the measured value of the particle concentration may be divided by ten or otherwise normalized to the original, unseparated sample).

FIG. 2-FIG. 4 illustrate various example PM₁₀ inlets of some embodiments that are generally cylindrical in design to minimize disturbance of wind flow around the sensor. These inlets also may be used with various PM sensors (e.g., a direct-reading PM_{2.5} sensor), especially those having low flow rates. Such an approach allows for the fractionation of particles into two particle size ranges, such as those below 10 μm AD at particular collection efficiency (e.g., fifty percent) and those sized above 2.5 μm AD at particular collection efficiency (e.g., fifty percent) allowing for the separate collection and measurement of PM_{c} and PM_{2.5} as well as PM₁₀, where PM₁₀ may be calculated by summing PM_{2.5} and PM_{c}. PM₁₀ may also be measured directly when a secondary fractionator is not used and particles sized 10 μm AD and less are directly collected on a sensor.

Some embodiments provided herein may maximize slope of the collection efficiency curve (e.g., having a target slope of 1.2 or lower) and minimize wall losses (e.g., with a target of less than ten percent loss). Dimensions outside the following specifications may result in a more gradual or less steep slope of the collection efficiency curve (reflecting a less clean separation between two size ranges) and an increase in wall losses. These dimensions have not been defined previously for the direct-reading PM2.5 mass sensor.

For round jet impactors, the collection jet diameter (D_{c}) and acceleration jet diameter (Dⱼ), have a target ratio (D_{c}/Dⱼ) between 1.2 and 1.5 (or, for improved performance, between 1.25 and 1.45). The jet spacing (Sⱼ) between the collection and acceleration jets may be between 1.2 and 1.5 (or, for improved performance, between 1.25 and 1.45) times Dⱼ. For example, a round jet having a Dⱼ equal to 200 μm, may have a target D_{c} of 270 μm (i.e., 1.35 times Dⱼ). The jet spacing, Sⱼ, may also have a target of 270 μm (i.e., 1.35 times Dⱼ). Impactors may typically be operated in laminar flow, having a Reynolds number (Reⱼ) less than 8,000. Some embodiments may operate with Reⱼ up to 80,000 or higher. Similar ratios apply for slit impactors based on a short side of the slit.

Once a design for a fractionator (e.g., a PM₁₀ inlet, PM_{2.5} fractionator, etc.) is physically defined, Stokes' law may provide general guidelines to adjust the dimensions of the components of the fractionator (e.g., jets and jet spacing) in order to obtain similar collection efficiency and cut point size at different flow rates using the same geometry. Once a Stokes' parameter (Stk) is determined for a given physical design of the impactor, the Stk₅₀ (Stokes number at fifty percent collection efficiency) may be used in the equation to calculate a new Dⱼ, from which D_{c} and Sⱼ may be derived. As a first approximation for round jet impactors, the cube root of the ratio of the flow rates may be multiplied by the initial Dⱼ to obtain the resultant Dⱼ as a function of flowrate.

FIG. 2 illustrates a front elevation view of a VI 200 of some embodiments used as an inlet for a direct-reading PM_{2.5} mass sensor at low flowrates (e.g., <50 mL/min) or other low flowrate PM or gas sensors (higher flowrates may apply depending on the application). As shown, the VI 200 may include a conical section (or “input port”) 210, a cover 220 (e.g., a rain guard and wind stabilizer), a body 230, acceleration jet 240, a first output 250, a collection cone 255, a second output 260, and a fan or pump 270. The VI 200 may serve as the primary particle size fractionator 110 of some embodiments.

The internal body 230 may be generally cylindrical and may house the various components of the VI 200. The cover 220 may be coupled to the body 230 in various appropriate ways and may include a screen and may prevent rain, insects, refuse, etc. from entering the VI 200.

The acceleration jet 240 may extend out from an output of the conical section 210 and may be cylindrically shaped, with a rounded shape at the connection point 245 between the acceleration jet 240 and the input port 210. Other shapes may apply.

The first output 250 (or “fine” output or “primary” output) may include particles smaller than the cut point size at a particular collection efficiency (e.g., fifty percent) and may be connected to a secondary particle size fractionator of some embodiments. The conical inner body 255 may include a sloped outer wall, as shown. The second output 260 (or “coarse” output or “secondary” output) may include particles larger than the cut point size and may be discarded or otherwise removed or transported to another fractionator. Because larger sized particles are removed by VI 200, the VI 200 may be very low maintenance.

A center line of each of the input port 210, acceleration jet 240, collection cone 255, and second output 260 may be aligned along axis 265, which runs along the center of VI inlet 200.

The fan or pump 270 may include, and/or be associated with, various electrical and/or electronic devices that may be able to control air flow through the VI 200.

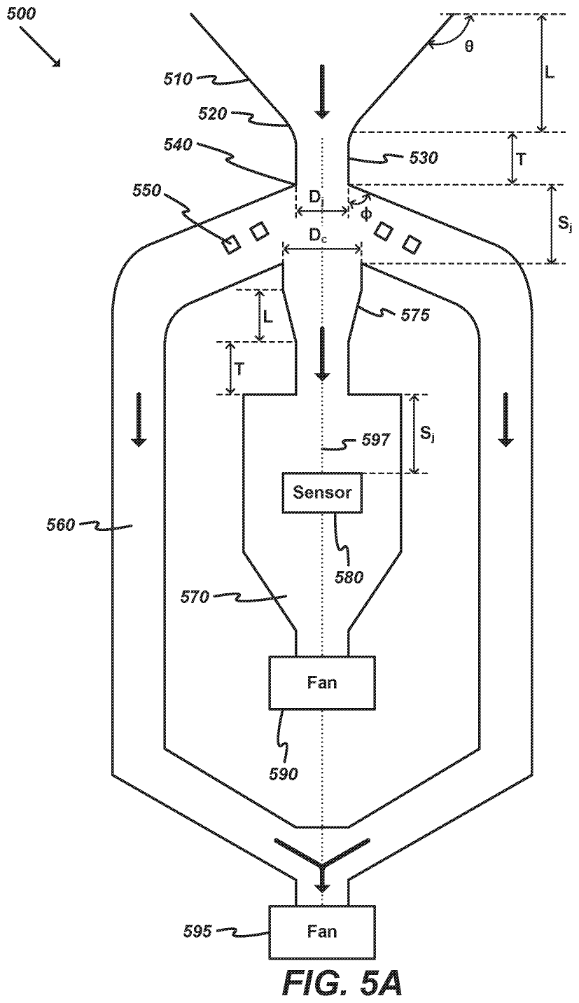

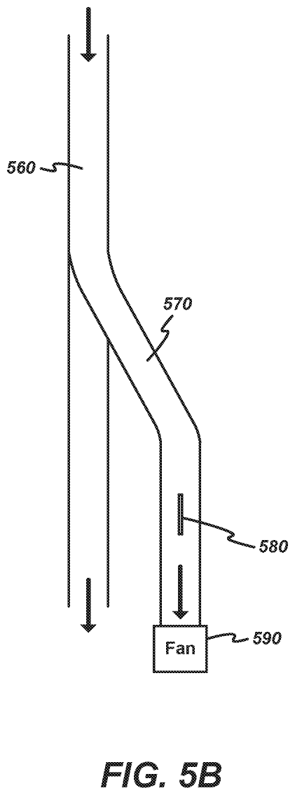

Such a VI 200, having a single fine particle channel as shown, may require only one or two sensing element resonators (e.g., for fine and coarse channels) and therefore uncertainty of the measurement in the collection of fine particles may be reduced by having only two flow rates to control, as shown, rather than three (such as in the example of FIG. 5A). In addition, uncertainty associated with the collection of particles on sensing elements associated with one channel may be reduced without requiring a minimum of two (or more, if in parallel in the channel in some embodiments) sensing element resonators in each channel. Some embodiments, such as those operating at a total flow rate of between 1 mL/min to 50 mL/min (or higher, depending on the application), may include only one jet. Alternatively, multiple jets may be included. In this example, the acceleration jet and collection jet each include single round nozzles, but some embodiments may include small rectangular nozzles (e.g., one hundred microns by two hundred microns) or long rectangular slits that encircle the entire fractionator (not shown). Some embodiments may include multiple nozzle pairs (each pair including an acceleration jet and a collection jet) operated at these low flow rates with the flow of air through each nozzle equal to the total flow divided by the number of nozzles.

Some embodiments of VI 200 may provide a cut point of 10 μm AD with fifty percent collection efficiency. Preferred relative and absolute sizing are provided for one exemplary embodiment of such a PM₁₀ inlet. The exemplary embodiment may have a flow rate of 50 mL/min. Ratios of the various dimensions for the exemplary embodiment are provided by equations (1) to (5) below:

T=1.5·Dⱼ (1)

Sⱼ=1.33·Dⱼ (2)

D_{c}=1.33·Dⱼ (3)

S_{c}=4·Dⱼ (4)

L=5.4·Dⱼ (5)

Further, the exemplary embodiment may be associated with target ratio ranges for round jet VIs as provided by equations (6) to (9) below:

1.2<Sⱼ/Dⱼ<1.5 (6)

1.2<D_{c}/Dⱼ<1.5 (7)

3<S_{c}/Dⱼ<8 (8)

3<L/Dⱼ<10 (9)

Similar ratios apply for slit impactors, based on the short side of the slit. For example, for a direct-reading PM_{2.5} VI, a rectangular slit of 200 μm by 100 μm may result in a collection jet rectangle of about 135 μm by 270 μm, with a spacing of 135 μm.

Specifically, the exemplary embodiment may utilize the following dimensions, at 50 mL/min: Dⱼ=464 μm, D_{c}=617 μm, Sⱼ=617 μm, T=696 μm, S_{c}=1856 μm, and L=2506 μm. Further, angle θ may be between forty-five and sixty degrees in some embodiments and angle ϕ may be between forty-five and sixty degrees in some embodiments. One of ordinary skill in the art will recognize that various ratios, dimensions, etc. that are given throughout the disclosure are provided for example purposes and different embodiments may be implemented with different specific dimensions, ratios, etc. than those provided. Furthermore, any provided dimensions, ratios, or other design parameters may be approximations, reflect rounded calculations, and/or otherwise differ from actual implementations as appropriate. Impactors, especially, may require empirical calibration in the laboratory with particles of known size and composition.

The smaller particles (i.e., particles sized less than the cut point) may follow the major flow 250 (e.g., eighty-five to ninety-six percent of particles) to the side channel and may be transported to a sensing element or secondary particle size fractionator.

For a particle size fractionator with a cut point of 10 μm AD, the smaller particles (i.e., those particles having less than 10 μm AD) may either be collected directly onto, for example, a quartz crystal, solid mounted resonator, or other resonator that can support collecting particles greater than 3 μm AD, for example, without loss of efficiency, a filter for collection and mass and chemical analysis in a laboratory, or to a secondary fractionator 130 to separately collect other particle sizes or size ranges smaller than the cut point. Other particle sensors (e.g., light scattering or light absorption) or gas sensors) may apply. Different embodiments may include different specific features, depending on the particle sizes of interest.

When the above-referenced exemplary embodiment is used as a primary particle size fractionator inlet 110, the particle size fractionator described below in reference to FIG. 5A and FIG. 5B may be used as a secondary particle size fractionator in some embodiments.

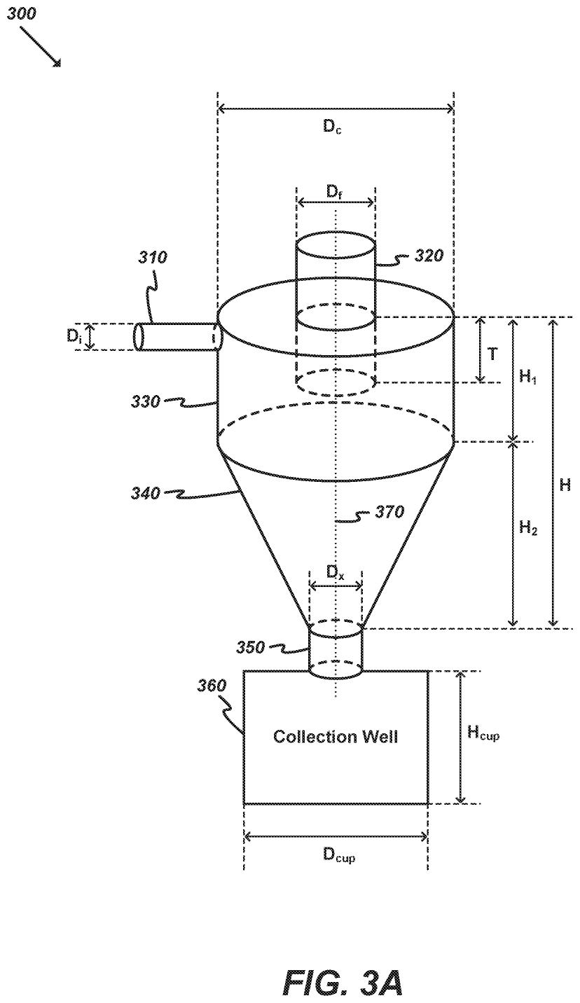

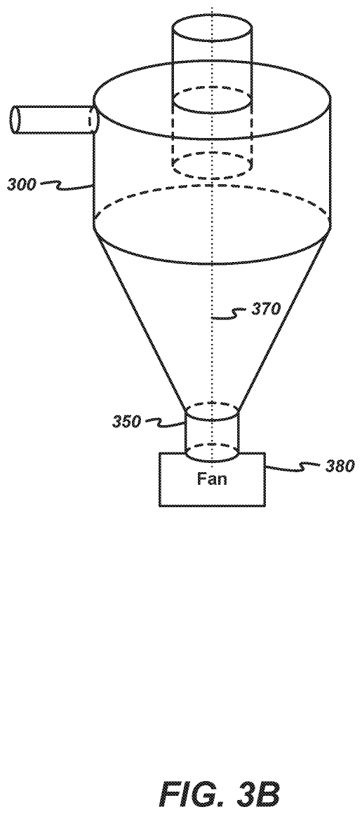

FIG. 3A illustrates a front elevation view of an inlet or primary fractionator 300 of some embodiments that includes an ultra-low flow rate cyclone. Such a fractionator may be operated between 1 mL/min and 50 mL/min, for example, where higher flow rates may apply depending on the application. As shown, the cyclone inlet 300 may include an input 310, a first output 320, a cylindrical body portion 330, a conical body portion 340, a second output 350, and a collection well 360.

As shown, the cylindrically-shaped first output 320 and second output 350 may have center lines along the same center axis 370 of the inlet 300, where air flow out of the first output 320 and second output 350 may be in opposite directions along axis 370. Likewise, cylindrical body portion 330 and conical body portion 340 may have center lines along the same axis 370.

Inlet 300 may be used as an inlet for the direct-reading PM_{2.5} mass sensor at low flowrates (e.g., less than 50 mL/min) or other low flowrate PM or gas sensors (higher flowrates may apply depending on the application), where such PM sensors may directly provide PM mass measurements in two size ranges.

Such a virtual cyclone would significantly extend the lifetime between periods when the collection-well would need to be cleaned because particles sized larger than the primary cut point would be removed from the well providing a very low maintenance inlet for PM or gas sensors. Particles transported through the first output 320 (i.e., particles sized less than the cut point) may be transported to a sensing element and may either be measured directly as PM₁₀ (for a cyclone with a 10 μm AD particle size cut point) onto a quartz crystal, SMR, or other resonator that can support collecting and measuring the mass of particles less than 10 μm AD without loss of efficiency or to a PM sensor based on light scattering. In some embodiments, particles transported through the first output 320 may be transported to a filter for collection and subsequent determination of mass and chemical analysis in a laboratory, or to a secondary fractionator of some embodiments.

FIG. 3B illustrates a front elevation view of an inlet or primary fractionator 300 of some embodiments that includes a fan or pump 380. Some percentage (e.g., four to fifteen percent) of the particles sized less than the cut point and all particles (at, for instance, 50% collection efficiency) sized greater than the cut point may be removed from the base 350 of the inlet or primary fractionator 300 through the fan or pump 380.

In some embodiments rather than removal of particles from the system, the primary output 320 may include a collection/sensing element that can measure particles greater than 2.5 μm AD (e.g., for a PM₁₀ inlet). In still other embodiments, at the primary output 320, some embodiments may include a second size fractionating device (e.g., a virtual cyclone or VI) where some percentage (e.g., four to fifteen percent) of the particles less than the cut point and all particles (at, for instance, 50% collection efficiency) may be further size fractionated, providing an additional size range. For example, virtual cyclone 300 may have a cut point of 10 μm AD and the secondary size fractionator may have a cut point of 2.5 μm AD. Thus, measured particle size ranges may include particles sized greater than PM₁₀ (via the sensing element at second output 350), greater than 2.5 μm AD (via the sensing element at the secondary output of the secondary particle size fractionator), and less than 2.5 μm AD, (via the sensing element at the primary output of secondary fractionator).

When the above-referenced exemplary embodiment inlet or primary fractionator 300 is used as inlet 110, the particle size fractionator described below in reference to FIG. 5A and FIG. 5B may be used as a secondary fractionator 130 to obtain additional separate particle size fractions below the cut point of the primary fractionator, in some embodiments.

Some embodiments of cyclone 300 may provide a cut point of 10 μm AD with fifty percent collection efficiency. Preferred relative and absolute sizing are provided for one exemplary embodiment of such a PM₁₀ inlet. The exemplary embodiment may have a flow rate of 50 mL/min. The exemplary embodiment may utilize the following dimensions: Dₓ=0.71 mm, Dᵢ=0.68 mm, D_{c}=2.82 mm, D_{f}=0.76 mm, T=0.99 mm, H₁=1.21 mm, H₂=3.19 mm, H=4.40 mm, D_{cup}=1.79 mm, and H_{cup}=2.46 mm.

Preferred relative and absolute sizing are provided for an additional exemplary embodiment of such a PM₁₀ inlet. The additional exemplary embodiment may have a flow rate of 55 mL/min. The additional exemplary embodiment may utilize the following dimensions: Dₓ=0.74 mm, Dᵢ=0.71 mm, D_{c}=2.97 mm, D_{f}=0.80 mm, T=1.04 mm, H₁=1.28 mm, H₂=3.36 mm, H=4.64 mm, D_{cup}=1.89 mm, and H_{cup}=2.59 mm.

Dimension of cyclone 300 may be specified by equations (10)-(19) below:

Dᵢ=0.24·D_{c} (10)

D_{f}=0.27·D_{c} (11)

Dₓ=0.25·D_{c} (12)

H=1.56·D_{c} (13)

H₁=0.43·D_{c} (14)

H₂=1.13·D_{c} (15)

T=0.35·D_{c} (16)

H_{cup}=0.87·D_{c} (17)

D_{cup}=0.635·D_{c} (18)

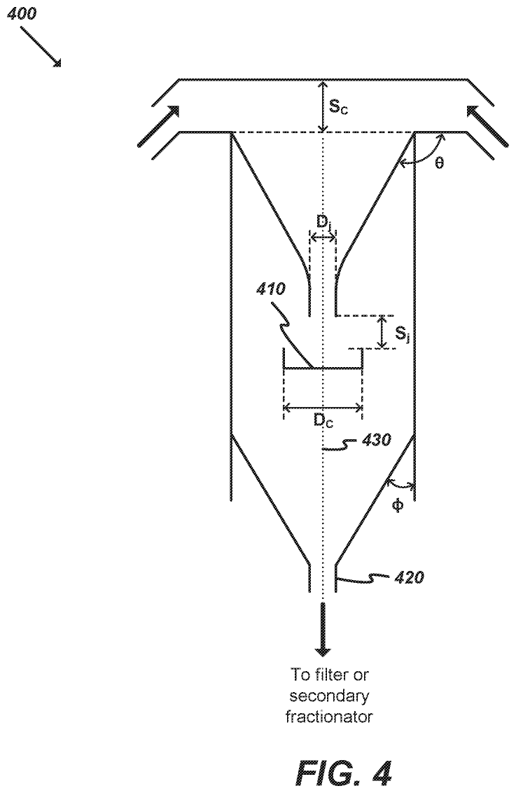

FIG. 4 illustrates a front elevation view of a real impactor 400 of some embodiments. Real impactor 400 may be used as an inlet for a direct-reading PM_{2.5} mass sensor or other low flow rate PM or gas sensors. Such a fractionator may be operated between 2 mL/min and 50 mL/min, for example. As shown, the impactor 400 may include a collection plate 410 and an output 420. The inlet, cover, body, and other elements may be similar to those described in reference to VI 200 above. As shown, the elements of inlet 400 may have center lines along the same center axis 430 of the inlet 400.

The collection cup 410 may remove particles greater than a specified cut point (e.g., greater than 10 μm) at some specified efficiency (e.g., fifty percent) and may be removable to easily allow for periodic cleaning. Collection cup 410 may be utilized when a real impactor 400 is positioned in a vertical orientation. For a real impactor 400 positioned in an alternative orientation (e.g., horizontal), collection cup 410 may be replaced by a flat plate such that particles greater than the cut point impact on the plate and fall by gravity to the bottom of the impactor, providing a low maintenance impactor.

In this example, the collection cup 410 may have dimensions for a PM₁₀ inlet, as specified by the ratios in equations (19) and (20) below:

Sⱼ=1.35·Dⱼ (19)

3·Dⱼ≤D_{c}≤8·Dⱼ (20)

Different embodiments may include different specific ratios or dimensions. Dimensions are approximate and are applicable for a real impactor of this type with a flow rate of 50 mL/min.

Particles smaller than the specified cut point may be removed through the bottom cone and may exit the output 420 to a sensing element and may either be measured directly (e.g., as PM₁₀), onto a quartz crystal, SMR, or other resonator that can support collecting particles up to 10 μm AD without loss of efficiency. Alternatively, the output 420 may transport particles to a filter for collection and determination of mass and chemical analysis in a laboratory, or to a secondary fractionator When the above-referenced exemplary embodiments is used as a primary particle size fractionator or inlet 110, the particle size fractionator described below in reference to FIG. 5A and FIG. 5B may be used as a secondary fractionator 130 in some embodiments to obtain two particle size fractions both less than the cut point of the primary fractionator.

FIG. 5A illustrates a front elevation view of a secondary fractionator 500 of some embodiments. FIG. 5B illustrates a side elevation view of secondary fractionator 500. In this example, the fractionator 500 is a VI. Such a fractionator may be operated between 1 mL/min and 50 mL/min (where higher flowrates may apply depending on the application). With the addition of a PM₁₀ inlet, such as the inlets described above in reference to FIG. 2-FIG. 4, prior to secondary fractionator 500, the fractionators in this example with a PM_{2.5} cut point may obtain particles in two size ranges—a fine fraction (e.g., less than 2.5 μm AD) and a coarse fraction between, for example, 10 μm AD and 2.5 μm AD. As shown, the fractionator 500 may include an input 510, a throat junction 520, an acceleration jet 530, a main channel junction 540, one or more sensor elements 550, at least one major channel 560, at least one minor channel 570, a collection jet 575, a direct measurement mass sensor element 580, and one or more fans or pumps 590-595.

A center line of each of the input 510, acceleration jet 530, collection jet 575, sensor 580, and fan 590 may be aligned along axis 597, which runs along the center of secondary fractionator 500.

Using the secondary fractionator 500 to remove and measure particles greater than the cut point (e.g., particles in the size range between PM₁₀ and PM_{2.5} for a PM₁₀ cut point primary fractionator and a PM_{2.5} secondary fractionator) allows the larger particles to be measured away from the direct-reading mass sensor 580 (e.g., a PM_{2.5} sensor). Such an approach allows measurement and easy maintenance for the larger size fraction (e.g., particles in the size range between PM₁₀ and PM_{2.5} for a PM₁₀ cut point primary fractionator and a PM_{2.5} secondary fractionator) passing to the minor channel 570 associated with the direct-reading PM mass sensor 580. Calculated Reynolds numbers may be below 1,500 such that the sensor 500 may operate in laminar flow.

Mass sensor element 580 may be a QC, SMR, or other resonator or light scattering sensor appropriate for measuring, for example, PM_{c} (e.g., particles greater than 2.5 μm AD). Channel 560 or channel 570 may provide an output that is able to be passed to additional fractionators, as appropriate.

Sensor elements 550 may be FBARs that collect fine particles (e.g., particles sized less than 2.5 μm AD). Larger particles may be collected on a quartz crystal, SMR, or other resonator that is outside the plane of the sensor body extending from the coarse minor channel 570 in some embodiments. Such an approach allows for the smallest possible VI, as quartz crystals are much larger than FBARs (as currently used in the direct-reading PM_{2.5} mass sensor). Sensor 580, being located outside the plane of the sensor body may be removable to allow for maintenance or replacement. The loading of the quartz crystal, SMR, or other resonator or light scattering sensor that allows for the collection and measurement of particles greater than 2.5 μm AD may be monitored until a maximum threshold is reached after which the resonator in the minor channel maybe replaced or cleaned.

Each major channel 560 of the virtual impactor may include multiple sensing elements 550 (e.g., FBARs) in series (as shown) and/or parallel for measurement of, for example, PM_{2.5} mass. Whether arranged serially or in parallel, multiple FBARs allow for simultaneous collection of additional material to provide for an improved limit of detection, when operated one at a time allow for an increase in the lifetime of the sensor 500, and if in series, allow for the determination of the collection efficiency of the sensor for particles less than 2.5 μm AD, and/or other sizes.

The example fractionator 500 has a general flow split of ninety percent into the major channels 560 and ten percent into the minor channel 570, for example. Some embodiments may have different ratios between the major and minor channel(s) (e.g., four percent of the flow to the minor channel 570 and ninety-six percent of the flow to the major channels 560). Some embodiments may include a VI with a single major channel as described above in reference to VI 200, with between ninety percent and ninety-six percent of the flow to the major channel, for example.

Dⱼ in this example, may range from 685 μm at 50 mL/min to 182 μm at 6 mL/min for a PM_{2.5} cut point. These values are approximate. Elements 200, 300, or 400 may serve as the PM₁₀ (or other appropriately sized) inlet 510 to fractionator 500. Coarse particles may be transported through the acceleration jet 530 to the collection jet 575 and may impact on a quartz crystal (QC), SMR, or other resonator 580 whose frequency changes as a direct function of the mass deposited. The minor channel 570 may be located inside the sensor body if using an FBAR for particles less than 2.5 μm if the cut point of the VI is below 2.5 μm AD (e.g., 1 μm AD or 0.1 μm AD), as the FBAR may not be efficient for particles greater than 2.5 μm AD. In some embodiments, as shown in FIG. 5B, the minor channel 570 may be located outside the sensor body with the mass sensing element 580 outside the plane of the sensor. Two advantages are gained when PM_{c} (e.g., particles greater than 2.5 μm AD and less than a size greater than 2.5 μm AD, such as 10 μm AD) is obtained outside the sensor body of some embodiments. First, the secondary particle size fractionator 500 may be smaller focusing on particles less than the cut point because a QC or SMR is larger than the FBAR and will not fit inside the particle size fractionator 500 as previously defined and secondly, it may be easier to remove the QC or SMR for cleaning if outside the minor channel. In either case, the QC, SMR, or other resonator may be able to be removed from the sensor 500 if the mass loading reaches a critical value (as determined based on a shift in frequency of the resonator). The sensor may be positioned in a vertical orientation to provide the steepest collection efficiency curve and to minimize wall loss of particles greater than about 3 μm AD. The sensor may be positioned in various other orientations (e.g., horizontal).

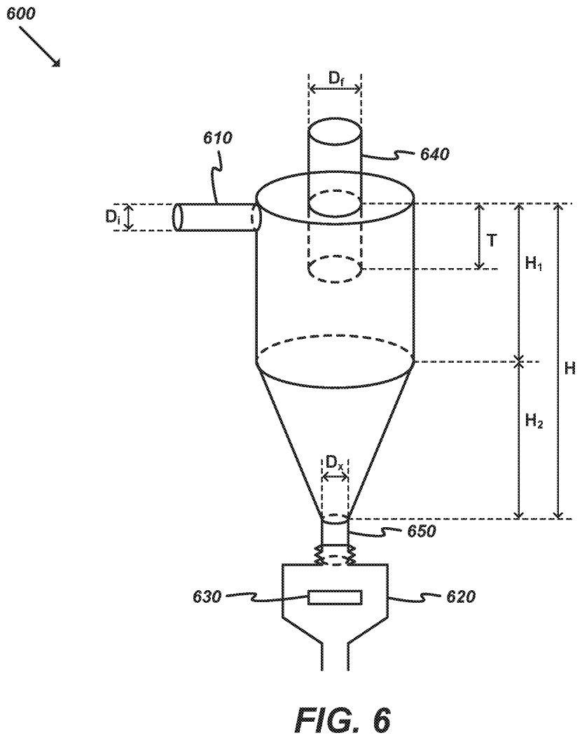

FIG. 6 illustrates a front elevation view of a virtual cyclone used as a secondary fractionator 600 of some embodiments. The components of fractionator 600 may be similar to those described above in reference to fractionator 300. Such components may be sized and/or otherwise optimized for particles less than 2.5 μm AD or other size fractions. Fractionator 600, for example having a PM_{2.5} μm cut point, may collect coarse particles, for example those between 10 μm AD and 2.5 μm AD using a PM₁₀ inlet such as those described above in reference to FIG. 2-FIG. 4.

Elements 200, 300, or 400 may serve as the PM₁₀ (or other appropriately sized) inlet to fractionator 600, where the inlet may be connected to input 610. In this example, a removable portion 620 includes a mass sensing element 630. The input flow rate for secondary particle size fractionator 600 may be 5.4 mL/min, and may be as high as 48 mL/min (i.e., ninety-six percent of flow through a primary channel 640) or as low as 0.85 mL/min (i.e., eighty-five percent of flow through the primary channel 640) for inlets 200 or 300 or as high as 50 mL/min to mL/min for inlet 400.

A QCM, SMR, or other resonator 630 may be located at the secondary output 650, as shown. PM_{c} mass may be obtained based on the frequency changes of the QCM, SMR, or other resonator as mass is directly deposited. PM_{c} mass may be obtained using light scattering measurements. The flow through removable portion 620 may be between four percent and fifteen percent of the total flow entering the secondary fractionator 600 at input 610. The QC, SMR, or other particle sensing element 630 may be housed in the removable portion 620 in order to allow easy maintenance via removal from the sensor 600 once the mass loading reaches a critical value (e.g., as determined based on the shift in frequency of the resonator).

In some embodiments, QCM 620 may be replaced with a light source such as a light-emitting diode (LED) and detector such as a photodetector, allowing PM_{c} mass to be determined by light scattering. Although indirect, light scattering has been shown to be a useful method to estimating PM₁₀ and PM_{2.5} concentrations in sensors and air quality monitors and PM_{c} mass may be obtained by difference. Use of light scattering may reduce maintenance but also may require assumptions about particle shape, refractive index, and density to obtain mass concentration. These assumptions have considerable uncertainty associated with their estimates over a range of particle sizes due to difference in chemical composition that effect their refractive index and density. Nevertheless, reasonable estimates (e.g., plus or minus forty percent) to a known reference have been obtained, improving to within ten percent when the light scattering sensor has been calibrated to the known reference. Temperature and relative humidity can have a significant impact on light scattering below 2.5 μm AD but less so above 2.5 μm AD.

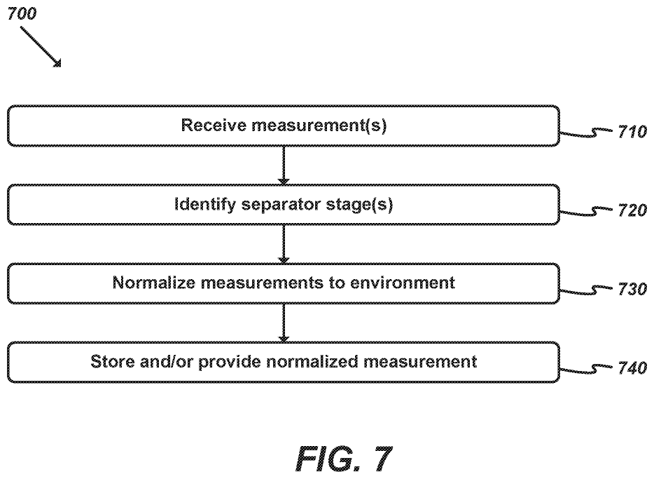

FIG. 7 illustrates an example process 700 that generates mass measurements for multiple particle sizes and/or size ranges. The process may be performed when an air quality sensing system 100 of some embodiments is powered on. In some embodiments, process 700 may be performed by air quality sensing system 100, and specifically by a resource such as particle collector and/or sensing element 120 or air quality sensor 130. Devices such as a user device (e.g., a smartphone, laptop, tablet, personal computer, wearable device, etc.) and/or server may perform complementary processes to process 700.

As shown, process 700 may include receiving (at 710) measurements data from one or more particle sensing elements. Process 700 may include identifying (at 720) a separator stage associated with each received measurement. Stages may include, for instance, a primary or inlet stage, secondary stage, tertiary stage, etc.

The process may include normalizing (at 730) the received measurements to the surrounding environment by adjusting mass readings for environmental variables (e.g., temperature, relative humidity, atmospheric pressure, etc.). Such normalization may scale measured mass based on concentration level at the stage in order to generate a measurement associated with the unprocessed environmental sample. In addition to normalizing the measurement(s), some embodiments may calculate differences in order to determine particle mass of particles within a size range (and/or otherwise calculate such information).

The process may include storing and/or providing (at 740) the normalized measurement data, raw collected sensing element data, and/or other appropriate data (e.g., sensor information such as type, location, model, etc.). Such data may be stored locally and/or stored at other system components (e.g., a user device, server, a local controller, etc.). Some embodiments may store collected data periodically, at regular intervals, and/or based on some other criteria (e.g., store collected data when a number of samples is exceeded, when a data request is received, etc.).

The data may be provided in various appropriate ways. For instance, some embodiments may provide such data via a user interface of the air quality sensing system 100 (e.g., by displaying test results, by providing a pass/fail indication, etc.). As another example, data may be provided via a user device or server. For instance, third-party researches may be able to retrieve data from a storage associated with server via the Internet.

One of ordinary skill in the art will recognize that process 700 may be implemented in various different ways without departing from the scope of the disclosure. For instance, the elements may be implemented in a different order than shown. As another example, some embodiments may include additional elements or omit various listed elements. Elements or sets of elements may be performed iteratively and/or based on satisfaction of some performance criteria. As still another example, non-dependent elements may be performed in parallel. In addition, process 700 (or elements thereof) may be divided in multiple sub-processes and/or included as part of a macro-process.

The processes and modules described above may be at least partially implemented as software processes that may be specified as one or more sets of instructions recorded on a non-transitory storage medium. These instructions may be executed by one or more computational element(s) (e.g., microprocessors, microcontrollers, digital signal processors (DSPs), application-specific integrated circuits (ASICs), field programmable gate arrays (FPGAs), other processors, etc.) that may be included in various appropriate devices in order to perform actions specified by the instructions.

As used herein, the terms “computer-readable medium” and “non-transitory storage medium” are entirely restricted to tangible, physical objects that store information in a form that is readable by electronic devices.

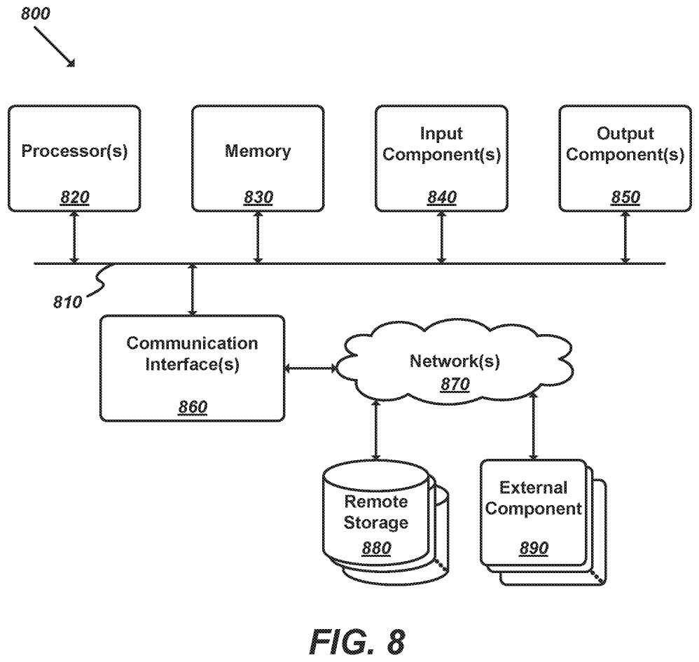

FIG. 8 illustrates a schematic block diagram of an exemplary device (or system or devices) 800 used to implement some embodiments. For example, the systems and devices described above in reference to FIG. 1-FIG. 6 may be at least partially implemented using device 800. As still another example, the process described in reference to FIG. 7 may be at least partially implemented using device 800.

Device 800 may be implemented using various appropriate elements and/or sub-devices. For instance, device 800 may be implemented using one or more personal computers (PCs), servers, mobile devices (e.g., smartphones), tablet devices, wearable devices, and/or any other appropriate devices. The various devices may work alone (e.g., device 800 may be implemented as a single smartphone) or in conjunction (e.g., some components of the device 800 may be provided by a mobile device while other components are provided by a server).

As shown, device 800 may include at least one communication bus 810, one or more processors 820, memory 830, input components 840, output components 850, and one or more communication interfaces 860.

Bus 810 may include various communication pathways that allow communication among the components of device 800. Processor 820 may include a processor, microprocessor, microcontroller, digital signal processor, logic circuitry, and/or other appropriate processing components that may be able to interpret and execute instructions and/or otherwise manipulate data. Memory 830 may include dynamic and/or non-volatile memory structures and/or devices that may store data and/or instructions for use by other components of device 800. Such a memory device 830 may include space within a single physical memory device or spread across multiple physical memory devices.

Input components 840 may include elements that allow a user to communicate information to the computer system and/or manipulate various operations of the system. The input components may include keyboards, cursor control devices, audio input devices and/or video input devices, touchscreens, motion sensors, etc. Output components 850 may include displays, touchscreens, audio elements such as speakers, indicators such as LEDs, printers, haptic or other sensory elements, etc. Some or all of the input and/or output components may be wirelessly or optically connected to the device 800.

Device 800 may include one or more communication interfaces 860 that are able to connect to one or more networks 870 or other communication pathways. For example, device 800 may be coupled to a web server on the Internet such that a web browser executing on device 800 may interact with the web server as a user interacts with an interface that operates in the web browser. Device 800 may be able to access one or more remote storages 880 and one or more external components 890 through the communication interface 860 and network 870. The communication interface(s) 860 may include one or more application programming interfaces (APIs) that may allow the device 800 to access remote systems and/or storages and also may allow remote systems and/or storages to access device 800 (or elements thereof).

It should be recognized by one of ordinary skill in the art that any or all of the components of computer system 800 may be used in conjunction with some embodiments. Moreover, one of ordinary skill in the art will appreciate that many other system configurations may also be used in conjunction with some embodiments or components of some embodiments.

In addition, while the examples shown may illustrate many individual modules as separate elements, one of ordinary skill in the art would recognize that these modules may be combined into a single functional block or element. One of ordinary skill in the art would also recognize that a single module may be divided into multiple modules.

Device 800 may perform various operations in response to processor 820 executing software instructions stored in a computer-readable medium, such as memory 830. Such operations may include manipulations of the output components 850 (e.g., display of information, haptic feedback, audio outputs, etc.), communication interface 860 (e.g., establishing a communication channel with another device or component, sending and/or receiving sets of messages, etc.), and/or other components of device 800.

The software instructions may be read into memory 830 from another computer-readable medium or from another device. The software instructions stored in memory 830 may cause processor 820 to perform processes described herein. Alternatively, hardwired circuitry and/or dedicated components (e.g., logic circuitry, ASICs, FPGAs, etc.) may be used in place of or in combination with software instructions to implement processes described herein. Thus, implementations described herein are not limited to any specific combination of hardware circuitry and software.

The actual software code or specialized control hardware used to implement an embodiment is not limiting of the embodiment. Thus, the operation and behavior of the embodiment has been described without reference to the specific software code, it being understood that software and control hardware may be implemented based on the description herein.

While certain connections or devices are shown, in practice additional, fewer, or different connections or devices may be used. Furthermore, while various devices and networks are shown separately, in practice the functionality of multiple devices may be provided by a single device or the functionality of one device may be provided by multiple devices. In addition, multiple instantiations of the illustrated networks may be included in a single network, or a particular network may include multiple networks. While some devices are shown as communicating with a network, some such devices may be incorporated, in whole or in part, as a part of the network.

Some implementations are described herein in conjunction with thresholds. To the extent that the term “greater than” (or similar terms) is used herein to describe a relationship of a value to a threshold, it is to be understood that the term “greater than or equal to” (or similar terms) could be similarly contemplated, even if not explicitly stated. Similarly, to the extent that the term “less than” (or similar terms) is used herein to describe a relationship of a value to a threshold, it is to be understood that the term “less than or equal to” (or similar terms) could be similarly contemplated, even if not explicitly stated. Further, the term “satisfying,” when used in relation to a threshold, may refer to “being greater than a threshold,” “being greater than or equal to a threshold,” “being less than a threshold,” “being less than or equal to a threshold,” or other similar terms, depending on the appropriate context.

No element, act, or instruction used in the present application should be construed as critical or essential unless explicitly described as such. An instance of the use of the term “and,” as used herein, does not necessarily preclude the interpretation that the phrase “and/or” was intended in that instance. Similarly, an instance of the use of the term “or,” as used herein, does not necessarily preclude the interpretation that the phrase “and/or” was intended in that instance. Also, as used herein, the article “a” is intended to include one or more items and may be used interchangeably with the phrase “one or more.” Where only one item is intended, the terms “one,” “single,” “only,” or similar language is used. Further, the phrase “based on” is intended to mean “based, at least in part, on” unless explicitly stated otherwise.

The foregoing relates to illustrative details of exemplary embodiments and modifications may be made without departing from the scope of the disclosure. Even though particular combinations of features are recited in the claims and/or disclosed in the specification, these combinations are not intended to limit the possible implementations of the disclosure. In fact, many of these features may be combined in ways not specifically recited in the claims and/or disclosed in the specification. For instance, although each dependent claim listed below may directly depend on only one other claim, the disclosure of the possible implementations includes each dependent claim in combination with every other claim in the claim set.

We claim:

1. A low-flow inlet for an air quality sensor, the low-flow inlet comprising:
an input;
a fractionator having a cut point;
a first output associated with particles sized less than the cut point; and
a second output associated with particles sized greater than the cut point

2. The low-flow inlet of claim 1, wherein the fractionator is a virtual impactor.

3. The low-flow inlet of claim 1, wherein the fractionator is a virtual cyclone.

4. The low-flow inlet of claim 1, wherein the fractionator is a cyclone.

5. The low-flow inlet of claim 1, wherein the fractionator is a real impactor.

6. The low-flow inlet of claim 1, wherein the first output is coupled to a sensing element.

7. The low-flow inlet of claim 1, wherein the first output is coupled to a secondary fractionator having a secondary cut point that is less than the cut point.

8. A low-flow inlet for an air quality sensor, the low-flow inlet comprising:
an input;
a fractionator having a cut point; and
a primary output associated with particles sized less than the cut point.

9. The low-flow inlet of claim 8, wherein the fractionator is a virtual impactor comprising:
an acceleration jet; and
a collection jet.

10. The low-flow inlet of claim 8, wherein the fractionator is a real impactor comprising:
an acceleration jet; and
a collection cup or a collection plate.

11. The low-flow inlet of claim 8, wherein the fractionator is a cyclone comprising:
a cylindrical body portion;
a conical body portion; and
a collection well.

12. The low-flow inlet of claim 8, wherein the fractionator is a virtual cyclone comprising:
a cylindrical body portion;
a conical body portion; and
an output fan or blower.

13. The low-flow inlet of claim 8, wherein the primary output is coupled to a sensing element.

14. The low-flow inlet of claim 8, wherein the primary output is coupled to a secondary fractionator having a secondary cut point that is less than the cut point.

15. A low-flow air quality sensor comprising:
a low-flow inlet that includes a primary fractionator associated having a primary cut point, the primary fractionator comprising a primary output associated with particles sized less than the primary cut point; and
a secondary fractionator coupled to the primary output of the primary fractionator, the secondary fractionator associated with a secondary cut point.

16. The low-flow air quality sensor of claim 15, wherein the secondary fractionator comprises:
an input port located along a center axis of the low-flow air quality sensor;
an acceleration jet located along the center axis of the low-flow air quality sensor; and
a collection jet located along the center axis of the low-flow air quality sensor.

17. The low-flow air quality sensor of claim 16, wherein
a ratio of a separation between an output of the acceleration jet and an input of the collection jet to a diameter of the acceleration jet is between 1.2 and 1.5, and
a ratio of a diameter of the collection jet to the diameter of the acceleration jet is between 1.2 and 1.5.

18. The low-flow air quality sensor of claim 15, wherein the secondary fractionator comprises a plurality of sensing elements arranged in series.

19. The low-flow air quality sensor of claim 18, wherein the secondary fractionator comprises a real impactor, cyclone, or virtual cyclone.

20. The low-flow air quality sensor of claim 15, wherein particles sized greater than the secondary cut point are removed from the secondary fractionator and measured with a first sensing element and particles sized less than the secondary cut point are measured with a second sensing element.
